(19) United States
(12) Patent Application Publication
(10) Pub. No.: US 2017/0127983 A1
(43) Pub. Date: May 11, 2017

(54) SYSTEMS AND METHODS FOR SAMPLING CALIBRATION OF NON-INVASIVE ANALYTE MEASUREMENTS

(71) Applicants: Massachusetts Institute of Technology, Cambridge, MA (US); Johns Hopkins University, Baltimore, MD (US)

(72) Inventors: Nicolas Spegazzini, Arlington, MA (US); Ishan Barman, Boston, MA (US); Narahara Chari Dingari, Princeton, NJ (US); Rishikesh Pandey, Arlington, MA (US); Jaqueline S. Soares, Ouro Preto (BR); Ramachandra Dasari, Shererville, IN (US)

(21) Appl. No.: 15/348,919

(22) Filed: Nov. 10, 2016

Related U.S. Application Data
(60) Provisional application No. 62/253,558, filed on Nov. 10, 2015.

(51) Int. Cl. A61B 5/1495; A61B 5/1455; A61B 5/145

(52) U.S. Cl. CPC A61B 5/1495; A61B 5/14532; A61B 5/1455; G01N 21/274

(57) ABSTRACT

Systems and methods of the present invention provide a calibration model that requires minimal information compared with prior techniques. These systems and methods represent the first generalized approach for combined treatment of spectroscopic measurements of a dynamic, mass-transfer system with the underlying kinetic model of said system. The technique can be applied to non-invasive glucose monitoring or monitoring of chemical reaction dynamics.

### RELATED APPLICATION

This application claims priority to U.S. Provisional Patent Application No. 62/253,558, filed Nov. 10, 2015, the entire contents of which is incorporated herein by reference.

### STATEMENT OF GOVERNMENT SUPPORT

This invention was made with Government support under Grant No. P41 EB015871 awarded by the National Institutes of Health. The Government has certain rights in the invention.

### BACKGROUND OF THE INVENTION

A host of measurement techniques have been developed to measure chemical composition or the presence of bio-analytes in a sample. For example, spectroscopic techniques can provide information about the presence of specific constituents based upon unique spectral signatures of each constituent. Unfortunately, samples are rarely pure enough to provide clean spectroscopic signals. The presence of background signal and noise from other molecules in the field of view and the dynamic nature of each sample pose challenges for those wishing to extract real-time information about constituent concentration. The dynamic nature of samples poses a particular problem in that the target constituent(s) can change over time or may enter or exit the field of view during a time-course of measurements.

The extraction of usable data from spectroscopic measurements that is directly correlated to constituent concentration is a primary goal of many systems. A typical approach to solving the problem of signal extraction in noisy and dynamically changing samples is calibration. Calibration is a linear comparison between the response of a constituent and the signal of the instrument (technique) to obtain quantitative information. For any technique, calibration is imperative for the quantification of a constituent. Without calibration there typically can be no quantification. Furthermore, given the complexity of the recorded spectral signals, identifying the presence and/or concentration of a particular constituent cannot be typically performed by monitoring a few peaks. The use of “multivariate analysis”, especially PLS or other established techniques, performs the necessary function of correlating the spectral measurements to the desired state variable (concentration). However, these techniques require significant training data to develop an accurate calibration model. In many systems and circumstances, this poses a significant challenge because of sample paucity or the undesirability of frequent perturbation to a dynamic system.

Specifically, the use of training data sets can create an “overtrained” model that is so specific to a particular sample that it cannot provide generalized predictions that properly apply to other data sets. In addition, trained models are unlikely to apply between samples or patients due to many confounding factors including, as but a few examples, sample morphology and hydration/solution state. Acquisition of a significant number of “gold standard” measurements cannot be performed in many systems without compromising the identity of the samples. For example, in a bioreactor, making multiple concentration measurements entails the withdrawal of small quantities of the fluid mixture, and such a withdrawal perturbs the reaction mixture. Likewise, for non-invasive blood glucose estimation, multiple concentration measurements require multiple finger pricks, and these are a source of substantial inconvenience to the diabetic patient. A method is urgently needed to initialize and calibrate a chemical or bio-analyte measurement technique, particularly in the field of bio-analyte measurements in the blood.

Blood constituent (analyte) monitoring forms a substantial component of medical diagnostics, ranging from critical-care to point-of-care testing. The concentration levels of these analytes are tightly controlled under normal circumstances and thus any deviation from the well-established ranges can be immediately correlated with an abnormality in body function. Formulation and advance of non-invasive, continuous measurement strategies for such analytes—particularly glucose in diabetic patients—is highly desirable, given the significant challenges and inconvenience associated with multiple blood withdrawals per day. Furthermore, such a measurement technology would significantly aid neonatal and ICU patient monitoring as well as screening for pre-diabetes and gestational diabetes. Currently, the latter pathological conditions are diagnosed via functional loading tests (e.g., the oral glucose tolerance test or OGTT), where the insulin action is monitored by discrete finger-prick measurements over the duration of a few hours following an initial glucose stimulus.

To address this unmet clinical need for non-invasive, continuous measurement of blood analytes, vibrational spectroscopy, especially infrared (IR) absorption and Raman excitation, has been proposed due to its ability to quantify the biochemical composition of the blood-tissue matrix without necessitating addition of exogenous labels. Raman spectroscopy, in particular, has been exploited due to its exquisite chemical specificity emanating from the characteristic frequency shifts of photons following an interaction with the matrix molecule(s). This specificity provides an inherent advantage in targeted analysis of a specific bioanalyte as the congestion among the broad overlapping features in IR absorption spectra often washes out the information of interest.

Despite promising measurements of clinically relevant analytes (e.g., glucose, urea and cholesterol) in aqueous solutions and whole blood samples, the translation of spectroscopic techniques to in vivo measurements in humans has proven to be challenging. The primary impediment to clinical translation has been attributed to sample-to-sample variability in optical properties, such as those due to variations in skin-layer thickness and hydration state, and in physiological characteristics than can change over time in a patient or can vary from patient to patient.

### SUMMARY OF THE INVENTION

Systems and methods of the present invention provide multivariate kinetic modeling of a mass transfer process to allow quantitative monitoring of the kinetics of materials. In some embodiments, systems and methods described herein provide a generalized approach for spectroscopic measurements of a dynamic, mass-transfer system with the underlying kinetic model of said system. The incorporation of the kinetic model into the calibration process enables a spectroscopic calibration methodology to measure mass transfer processes.

Systems and methods of the present invention provide the ability to non-invasively measure analyte concentration levels in a subject. An exemplary system includes a spectroscopic measurement device to acquire spectral data and a computing device to process the data. The computing device applies a calibration approach including a kinetic model to predict concentration levels of an analyte based on components of the spectral data.

Preferred embodiments of the invention employ a computing device that is configured to execute a sequence of stored instructions to compute a characteristic of an analyte of interest in a biological system. A data processor connected to a memory can be used to compute the concentration of an analyte, for example, where the concentration can vary at a selected location in a biological system as a function of time.

In the present disclosure, an analytical formulation enables spectroscopy-based prediction of analyte information without necessitating reference concentration information for the development of the calibration model. The proposed framework is hereafter referred to as improved concentration independent calibration. The approach solves the inverse concentration estimation problem by incorporating the kinetic model of the system to guide spectroscopy-based concentration estimates. In other words, the kinetic model of the process provides a guide to the “missing” concentration piece of the inverse problem of concentration estimation. The fundamental principles of the method can be used for any spectroscopy-based quantification measurement and require the use of minimal information compared with current techniques to develop a calibration model.

Aspects of the present disclosure introduce multivariate kinetic modeling of a mass transfer model or, put simply, quantitative monitoring of the kinetics of one or more species where a mass transfer process occurs. A mass transfer process is the net movement of a substance or element from a first location to a second location. Certain embodiments of the present invention provide a mass transfer model where a chemical reaction may or may not occur.

Certain embodiments of the present invention focus on the application of the analytic framework to dynamic mass transfer processes in the human body such as non-invasive glucose monitoring. There are numerous mass transfer processes that occur in the human body that can be measured by vibrational spectral techniques such as NIR or Raman spectroscopy. These can include cellular transfer across barriers in an animal body or movement of cells in the vascular system. Within the framework, it is possible to characterize the physiological lag between the blood and interstitial fluid (ISF) glucose concentrations using a two-compartment mass transfer framework to model the analyte transport. Minimization of the spectral information and the output of the kinetic model is then pursued in the concentration domain. The spectroscopic calibration step is executed inside the kinetic parameter estimation loop in an iterative fashion. Such an approach considerably alleviates the rigidity associated with prior methods that sought to determine a simultaneous solution to the kinetic modeling and the spectroscopic calibration components.

Using concentration datasets obtained from a series of OGTTs in human subjects, embodiments of the present invention demonstrate the potential of the approach in estimating blood glucose concentrations. Estimates using the approach as described in greater detail below conform more closely to the measured values in relation to predictions computed from conventional PLS calibration, which show larger deviations. Aspects of the present invention provide quantitative insights into each subject's specific physiological lag characteristics to offer a new tool for the personalized assessment of diabetes onset and progression. Embodiments according to this invention are well-suited for clinical practice where obtaining intermediate concentration information is always challenging and often impossible.

Embodiments of the present application apply the powerful idea of indirect implicit calibration to quantitative biological spectroscopy. Spectroscopy-based inference of concentration of system constituents belongs to a class of inverse, ill-posed problems in the sense that there can be multiple solutions that are consistent with the measured data. Additionally, tracking the temporal evolution of a constituent necessitates analysis of the spectral time series often incorporating differential conservation equations and algebraic constitutive equations into the spectroscopic calibration framework. For example, continuous spectroscopy-based non-invasive glucose monitoring reflects the physiological dynamics of glucose transport between the blood and ISF compartments. Specifically, the time lag between the two glucose levels gives rise to an inconsistency in classical spectroscopic calibration models as the spectroscopic measurements primarily probe ISF glucose while blood glucose values are used as reference inputs. This problem is particularly exacerbated when measurements are performed during rapid changes in glucose levels such as immediately after a meal ingestion (as is the case for OGTT) or insulin administration. To address this important problem and related classes of monitoring applications, the residual between two concentration profiles is minimized. The profile computed from the kinetic model and that obtained from transformation of the spectral information is used to determine the concentration of the selected analyte.

To gainfully employ spectroscopic techniques in bioanalyte concentration prediction, chemometric methods, such as partial least squares (PLS) regression and support vector regression (SVR), have been employed to develop calibration models from representative samples. The multivariate calibration models are then used in combination with the spectrum acquired from a prospective sample to compute the bioanalyte concentration in that sample.

In view of the substantial inter-person variance in spectroscopic measurements, an alternative solution to establish the potential of vibrational spectroscopy is to perform time-lapse measurements (in a continuous or semi-continuous manner) on a single individual. Specifically, it would be beneficial to obtain a temporal evolution of the concentration profile solely from spectral acquisitions without resorting to (intermediate) concentration measurements. Such a method allows for minimum sample perturbation be it in a biomedical setting or in chemical reaction monitoring. Although the utility of such a protocol, which can function with little or no concentration information, is indisputable, there has been a lack of analytic frameworks that can operate solely based on the acquired spectroscopic and sample-specific kinetic information.

### BRIEF DESCRIPTION OF THE DRAWINGS

FIGS. 1A and 1B depict methods of measuring an analyte according to embodiments of the present invention.

FIG. 2 depicts a schematic illustration of light interacting with a tissue according to systems and methods of the present invention.

FIG. 3 depicts a representative Raman spectrum acquired according to devices and methods of the present invention described herein.

FIG. 4 depicts a series of analyte concentration measurements obtained according to embodiments of the present invention.

FIG. 5 depicts a Clarke Error Grid according to embodiments of the present invention.

FIG. 6 depicts tabular data including prediction results for embodiments of the present invention.

FIG. 7 depicts tabular data including performance characteristics for embodiments of the present invention.

FIG. 8 depicts a Bland-Altman plot of the difference in analyte concentrations (method—model predicted) for two models according to embodiments of the present invention.

FIG. 9 depicts a method of non-invasively measuring blood glucose concentration according to various embodiments of the present invention.

FIG. 10 depicts a system for obtaining and calibrating non-invasive bio-analyte concentration measurements according to various embodiments of the present invention.

FIG. 11 depicts a system for obtaining and calibrating non-invasive bio-analyte concentration measurements in a transmission orientation according to various embodiments of the present invention.

FIG. 12A depicts a longitudinal view of an apparatus including a probe for measuring tissue in accordance with a preferred embodiment of the present invention.

FIG. 12B depicts a transverse view of the probe illustrated in FIG. 12A in accordance with a preferred embodiment of the present invention.

FIG. 12C depicts a longitudinal view of an apparatus including a probe for measuring tissue in accordance with an embodiment of the present invention.

FIGS. 13A and 13B illustrates end views of a multi-mode probe and a single-mode probe, respectively, in accordance with the invention.

FIGS. 14A and 14B depicts forward looking probes with a ball lens and a half-ball lens, respectively in accordance with an embodiment of the present invention.

FIG. 15 depicts a side cross-sectional view of a side looking probe in accordance an embodiment of the present invention.

FIG. 16 depicts a method of measuring an analyte according to various embodiments of the present invention.

### DETAILED DESCRIPTION OF THE INVENTION

The present invention relates to spectroscopic quantification methods that require reduced information as compared with current techniques to develop a calibration model. Although the methodology is widely applicable to a variety of measurement techniques and samples, it is described in detail in the present disclosure as applied to non-invasive glucose monitoring for simplicity. However, it is to be understood that the approach is in no way limited to a particular field or application.

Vibrational spectroscopy has emerged as a promising tool for non-invasive, multiplexed measurement of blood constituents—an outstanding problem in biophotonics. Embodiments of the present invention include a novel analytical framework that enables spectroscopy-based longitudinal tracking of chemical concentration without necessitating extensive a priori concentration information. The principal idea is to employ a concentration space transformation acquired from the spectral information, where these estimates are used together with the concentration profiles generated from the system kinetic model. In addition to applications in blood glucose monitoring by Raman spectroscopy, the systems and method presented herein are efficacious in many scenarios compared to conventional calibration methods. Specifically, the present approach can exhibit a 35% reduction in error over partial least squares regression when applied to a dataset acquired from human subjects undergoing glucose tolerance tests. Improved predictions can offer a new route for screening gestational diabetes and can open doors for continuous process monitoring without sample perturbation at intermediate time points.

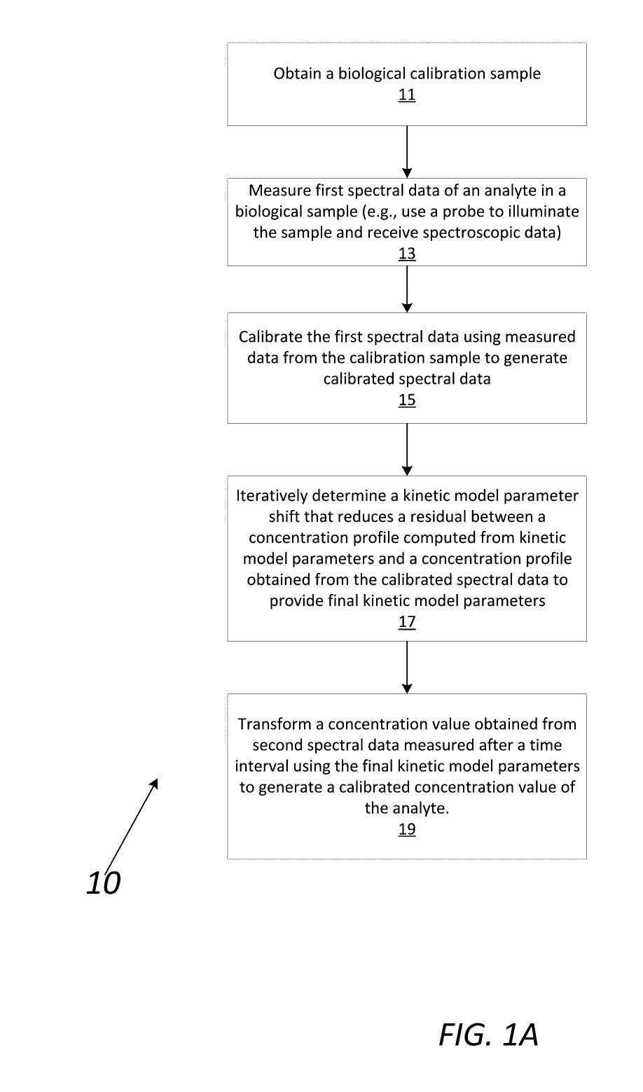

FIGS. 1A and 1B depict methods 10, 20 for measuring an analyte according to various embodiments. In step 11, a calibration sample from a patient is obtained. In step 13, first spectral data of an analyte in the sample is measured. In step 15, the first spectral data is calibrated using measured data from the calibration sample to generate calibrated spectral data.

In step 17 of the method 10, a kinetic model parameter shift that reduces a residual between a concentration profile computed from kinetic model parameters and a concentration profile obtained from the calibrated spectral data is iteratively determined to provide final kinetic model parameters. In step 19, a concentration value obtained from second spectral data measured after a time interval is transformed using the final kinetic model parameters to generate a calibrated concentration value of the analyte.

In step 11, obtaining a calibration sample can include a multitude of methodologies such as withdrawal of blood from a patient. In some embodiments, the obtained calibration sample can be a “gold standard” calibration sample known within the scientific or medical communities.

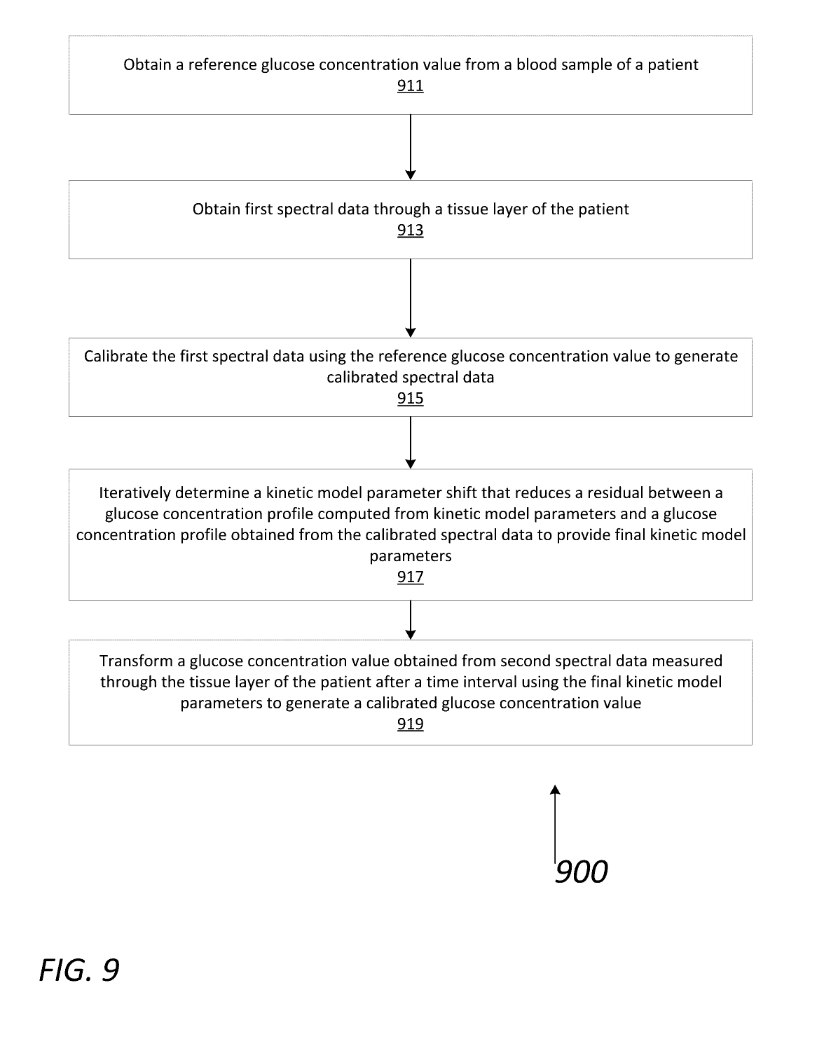

In step 13, first spectral data of an analyte in the sample can be measured, as only one example of many, using a spectroscopic probe as will be described in greater detail with respect to FIGS. 10-15 of this application.

In step 15, the first spectral data can be calibrated using measured data from the calibration sample to generate calibrated spectral data by, for example, noting the absolute values of and relationships between specific spectral features at a specific calibrated value of the analyte concentration obtained from the calibration sample as described in greater detail below. As shown in step 14 of FIG. 1B, a least-squares method such as singular value decomposition (SVD), partial least-squares (PLS), or principal component regression (PCR) can be employed to isolate relevant time-trace information in the first spectral data.

In step 17, a kinetic model parameter shift that reduces a residual between a concentration profile computed from kinetic model parameters and a concentration profile obtained from the calibrated spectral data can be iteratively determined to provide final kinetic model parameters by, for example but not limited to, employing a kinetic model methodology as described in greater detail below. As shown in FIG. 1B, a differential equation representing a kinetic model can be integrated to enable calculation of a concentration profile computed from the kinetic model parameters (step 16). In addition, a residual matrix can be calculated as a difference between a concentration profile computed from the kinetic model and a concentration profile obtained from the calibrated spectral data (step 18).

As shown in FIG. 1B, iterative determination of the kinetic model parameter shift may also include calculating a Jacobian by unfolding the residual matrix into a long vector and calculating a forward finite difference (step 22). The Jacobian may be used to generate updated kinetic model parameters for the next iteration (step 24). The method 20 further includes calculating a new residual matrix using updated kinetic model parameters and comparing to the previous residual matrix. In some embodiments, a decision step 27 includes comparison of the new residual matrix to a threshold value. If the value of the revised residual matrix has changed by equal to or greater than a threshold value, the method 20 continues to iterate. If the value of the revised residual matrix has changed by less than a threshold value, the last calculated kinetic model parameters become the selected or final kinetic model parameters.

In step 19, transformation of a concentration value obtained from second spectral data measured after a time interval using the selected kinetic model parameters to generate a calibrated concentration value of the analyte may be performed, for example but is not limited to, using the techniques described in greater detail below. In an exemplary embodiment as shown in FIG. 1B, the transformation may be carried out by computing the dot product of a regression matrix calculated with the selected kinetic model parameters and a concentration profile computed from second spectral data obtained after a time interval to generate a calibrated concentration value (step 28).

In accordance with the preferred systems and methods described herein, calibration of the acquired spectra is performed using concentrations calculated with iteratively improved kinetic parameter(s). Thus, the approach does not require repeated measurements of reference concentration values as detailed below. In various embodiments, the calculated concentrations, Ĉ, are considered to be “measured” variables and determination of the residual is performed in concentration units. This technique represents a multivariate calibration framework with “floating data”. Using concentration values Ĉ (a function of the kinetic parameters, k) and the recorded calibration spectral matrix Y, one can compute the corresponding regression matrix B using the least-squares solution to equation (1):

Ĉ=YB+E (1)

where E denotes the noise (error) in the measurements.

Given the underdetermined nature of the system (i.e., it has more variables, e.g., wavelengths, than equations, e.g., number of calibration data points), solution of the above equation implies calculation of a suitable pseudo-inverse of Y, such that \hat{B}=Y⁺Ĉ where \hat{B} represents the regression matrix estimate. The regression matrix estimate can be obtained using singular value decomposition (SVD), partial least squares (PLS) or principal component regression (PCR). The calibrated concentration profile Ĉ_{cal} is then determined by substitution in equation (1):

Ĉ_{cal}=YY⁺Ĉ (2)

This formulation can be employed to iteratively obtain the estimates of the kinetic parameters, k, by minimizing the following residual:

Q=∥Ĉ−Ĉ_{cal}∥=∥Ĉ−YY⁺Ĉ∥=∥(I−YY⁺)Ĉ∥ (3)

Equation (3), notably, defines the residual Q as a function of two altogether different concentration profiles: Ĉ, the concentrations computed based on the conservation equations governing the dynamic process; and Ĉ_{cal}, the spectroscopy-based concentration estimates obtained from the calibration step. Both concentrations are dependent on the current value of the kinetic parameters.

In order to reduce the impact of spectral baseline fluctuations and improve the contribution of each component of the spectral data during fitting, SVD of the spectral dataset Y is used to isolate the important time-trace information. The specific SVD procedure and its ability to alleviate the pernicious effect of baseline shifts are described further below. Reducing Y to Y=UΣV* (where U is the abstract time-trace matrix of concentration information, Σ is the diagonal matrix containing the singular values and V* is the abstract matrix of basis spectra), and replacing it in equation (3) provides:

Q=∥(I−UU^{T})Ĉ∥ (4)

Equation (4) represents the general framework for analysis of any time-resolved spectral data recorded from a dynamic system.

There are many possible systems in which application of embodiments of the present invention will lead to improved measurement calibration. In general, the formalism can be adapted to any system that may be modeled using a mass transfer equation. In these systems, a constituent or analyte of interest moves from a first position to a second position and generally crosses a barrier in so doing. Physiological examples include, but are not limited to, movement of constituents or analytes across the blood-brain barrier, kidney and liver filtration mechanisms, cellular exchange mechanisms, and exchange between blood vessels and surrounding anatomical compartments containing interstitial fluid. Anatomical sites that exhibit mass transfer of constituents or analytes include the eye, liver, kidney, brain, blood, stomach, lungs (e.g., gas exchange), and most other organs. Elements of mass transfer can occur during cancer metastasis. Moreover, mass transfer can occur in systems outside the body such as bioreactive vessels containing support media where cells may exchange constituents or analytes with their surroundings or be transported themselves as in, e.g., a flow cytometer.

Embodiments of the present invention can be used to calibrate analyte measurements over time. A single reference sample measurement taken at an initial time can continue to produce well-calibrated measurements according to the present invention at later times. According to various embodiments, the reference calibration sample can preferably be used to calibrate measurements up to one day later, more preferably 5 days later, or more preferably still up to 10 days (at least 240 hours) later or longer. In some embodiments, the final kinetic model parameters determined using the biological calibration sample can be used to transform the concentration value of second, third, or even larger-numbered spectral data measurements obtained after prescribed time intervals.

According to embodiments of the present invention, the solution formalism can be specialized for spectroscopy-based non-invasive monitoring of blood glucose. In such a formalism, the modeled concentration (Ĉ) and regression (\hat{B}) matrices are replaced by the corresponding ISF glucose-specific vectors (ĉ_{ISF}, \hat{b}_{ISF}). Because the acquired spectral data are representative of the ISF glucose concentrations, this replacement ensures consistency in the developed calibration models. To further remove ambiguity in the inversion problem and to rule out unphysical and implausible solutions, a secondary convex goal may be added by means of a regularization parameter. This added goal ensures that the minimization procedure converges on a robust solution in the sense that small variations in the spectral dataset do not cause large variations in the computed kinetic parameters, k, and the resultant regression matrix. This formalism is captured in equation (5):

Q_{reg}=∥[I−UU^{T})ĉ_{ISF}]∥²+λ∥k∥²→min (5)

where ĉ_{ISF} is assessed from the reverse form of the mass conservation-based model that governs the blood and ISF glucose relationship as described in greater detail below. The residual of equation (5), Q_{reg}, is minimized using the Newton-Gauss-Levenberg/Marquardt (NGL/M) algorithm for identification of the preferred kinetic parameters, kₒₚₜ.

The gradient-based Newton-Gauss-Levenberg/Marquardt algorithm (NGL/M) is used to solve the non-linear regression problem (equation (5)) obtained by employing the present approach. In particular, the sum of squares is calculated from Q and is used as the objective function to be minimized by iteratively altering the kinetic parameter set, k. The NGL/M algorithm provides the means to achieve this local minimum. Briefly, the aim of the NGL/M method is to determine a vector of parameter shifts (Δk), Δk=k_{new}−k_{old}, that moves Q(k₀+Δk) towards zero, where k₀ is the vector of initial parameter estimates.

Because any gradient-based method requires the calculation of a Jacobian matrix, i.e., the matrix of first partial derivatives of the residuals Q with respect to k, and such computation in this case leads to a three dimensional Jacobian, it is convenient to unfold Q into a long vector q (m.n×1). Then, the corresponding Jacobian J_{Q} can be calculated by a forward finite difference, as illustrated by equation (6):

$${J}_{Q} = ( \frac{\partial q}{\partial k} ) with ( \frac{\partial q}{\partial {k}_{i}} ) \approx \frac{q ( k + δ {k}_{i} ) - q ( k )}{δ {k}_{i}} \qquad ( 6 )$$

Here, kᵢ is the i^{th} kinetic parameter (rate constant of the dynamic process) and δkᵢ the finite difference applied to the i^{th} rate constant. This Jacobian matrix is used by the NGL/M algorithm to iteratively shift k towards an optimum. In particular, the vector containing the intrinsic rate constants, k_{new}, is updated from the previous vector, k_{old}, in the following manner:

k_{new}=k_{old}+(J_{Q}^{T}J_{Q}+∈·I)⁻¹·J_{Q}^{T}·q(k₀) (7)

where J_{Q} is the Jacobian matrix of Q residuals at k_{old}, I is the identity matrix, E is a scalar that assures that Q(k_{new})≦Q(k_{old}) and makes the minimization algorithm immune to possible singularities of (J_{Q}^{T}J_{Q})⁻¹.

Convergence is estimated by the criterion of change in Q by less than 10⁻⁵ in successive iterations. Conversely, the iteration process is aborted if convergence is not obtained after 1000 iterations. In order to validate successful convergence, it was further confirmed that the solution obtained did not vary when 1000 successful (i.e., not aborted) minimizations starting from randomly generated initial guesses for the rate constants were tested. This indicates that Q has a unique well-defined minimum in the physical domain, as expected for the intrinsic rate constants.

Solution of equation (5) yields the set of optimal ISF glucose concentrations (via kₒₚₜ), which in turn can be used to calculate the ISF glucose-specific regression vector \hat{b}_{ISF}. Using this regression vector in conjunction with the spectrum measured at the prediction time point (s_{pred}), one can predict the ISF glucose concentration:

ĉ_{ISF,pred}=(s_{pred})^{T}\hat{b}_{ISF} (8)

The set of predicted ISF glucose concentrations can be transformed using the forward form of the physiological glucose dynamics model and knowledge of the kinetic parameters kₒₚₜ to construct the corresponding blood glucose estimates.

The transport of glucose from the blood to the ISF compartment occurs by a diffusion process across an established concentration gradient. This process can be mathematically written in the following form for the glucose component in the ISF space:

$$\frac{d}{d t} ( {V}_{ISF} {c}_{ISF} ) = {k}_{21} {V}_{BG} {c}_{BG} - ( {k}_{12} + {k}_{02} ) {V}_{ISF} {c}_{ISF} \qquad ( 9 )$$

where c_{BG}, c_{ISF} are the concentrations of glucose in the blood and ISF compartments, respectively; V_{BG}, V_{ISF} are the volume of blood and ISF in the probed region; k₂₁ and k₁₂ are the forward and reverse flux rates for glucose transport across the capillaries; and k₀₂ is the rate of glucose uptake into the surrounding tissue. This equation can be re-written in the following form by reducing the additional parameters into a two-parameter (k₁, k₂) system:

$$\frac{d}{d t} ( {c}_{ISF} ) = {k}_{1} {c}_{BG} - {k}_{2} {c}_{ISF} \qquad ( 10 )$$

Re-arranging equation (10) forms the forward model that is used to compute the blood glucose concentrations based on the ISF glucose values and knowledge of the system parameters. Additionally, integrating equation (10) provides the reverse form of the model, which is plugged into equation (5) that in turn performs minimization of the objective function.

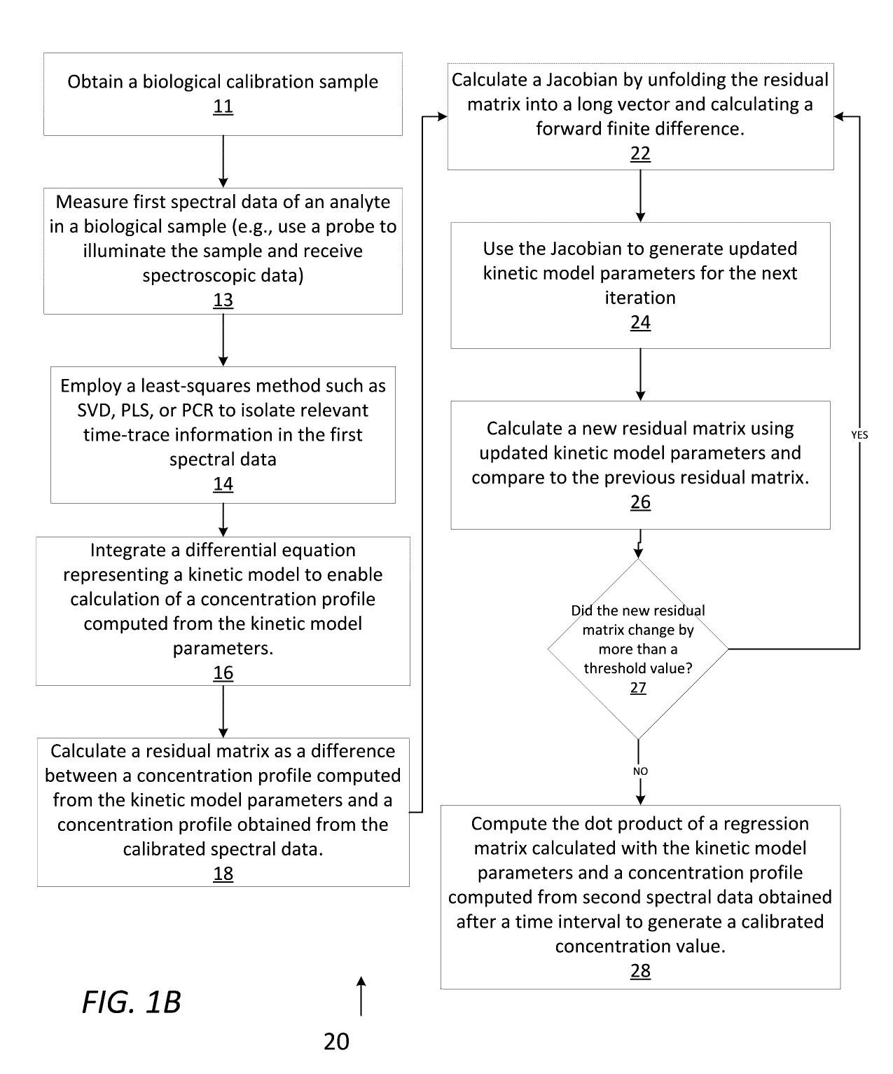

To illustrate the capability of the present methodology to predict concentrations from time-resolved spectra, clinical datasets comprised of blood glucose concentrations and Raman spectra are used. These datasets were collected from healthy human volunteers undergoing OGTT. In the present approach, Raman spectroscopy was chosen to evaluate the efficacy of the proposed methodology. However, the present approach can be utilized with other vibrational spectroscopic approaches, such as NIR absorption, in other embodiments. Such embodiments do not utilize the Raman filter probes as described herein, but utilize broadband infrared light sources and variable wavelength (700 nm to 2500 nm) sources or utilize a cadmium telluride detector array to detect the region of the electromagnetic spectrum in one or more channels as needed for the specific application. For near-infrared absorption, a fiber optic cable can be used to deliver light from a quartz halogen lamp or light emitting diodes (LEDs) depending on the spectral stability and power requirements. Silicon or InGaAs detectors can be used depending on the wavelength range being utilized. Optical coherence tomography (OCT) can be used for transmission measurements of tissue using near-infrared wavelengths. Raman spectra were recorded at regular 5 min intervals from the forearms of these volunteers. FIG. 2 schematically illustrates the spectroscopic measurement process and shows how the photons interact with the glucose molecules in the two distinct compartments.

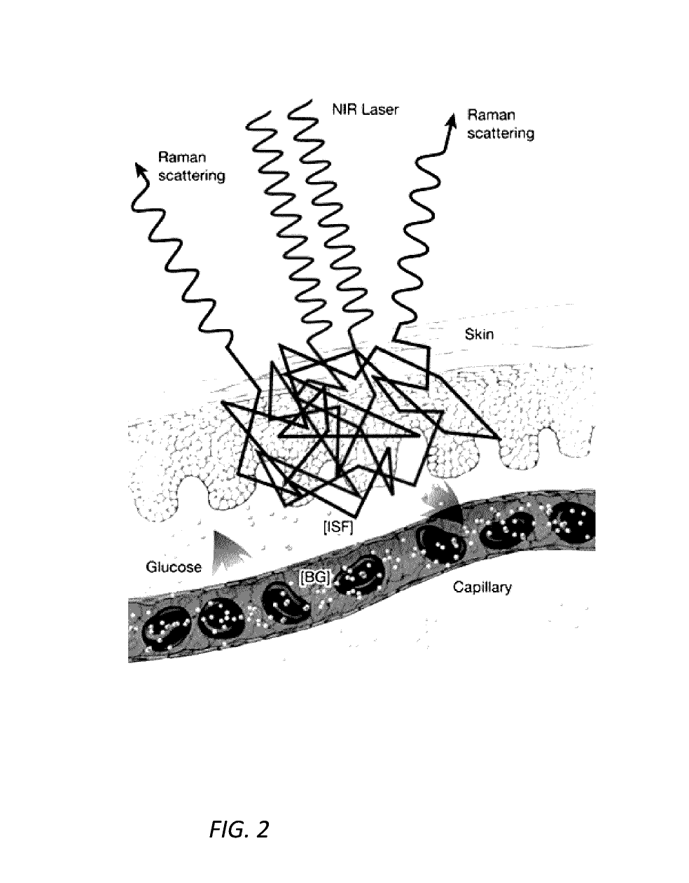

FIG. 3 shows the mean and ±1 standard deviation (SD) of representative Raman spectra acquired from a human patient undergoing an OGTT. In various embodiments, spectroscopic data may be acquired from a variety of locations on the human body including, but not limited to, the forearm, the hand, the thenar fold (skin fold between thumb and forefinger), the auricle of the ear, the earlobe, the eye, or the tympanic membrane. A system for measurement of spectroscopic data from the tympanic membrane is described in an article (Valdez, Tulio A., et al. “Multiwavelength fluorescence otoscope for video-rate chemical imaging of middle ear pathology.” Analytical chemistry 86.20 (2014): 10454-10460) and in U.S. Provisional Patent Application No. 62/218,947 entitled “SYSTEMS AND METHODS FOR DIAGNOSIS OF MIDDLE EAR CONDITIONS AND DETECTION OF ANALYTES IN THE TYMPANIC MEMBRANE” filed Sep. 15, 2015, and U.S. patent application Ser. No. 15/267,057, filed Sep. 15, 2016, the entire contents of the above publication and patent applications being incorporated herein by reference. In particular, the methods described in the above-mentioned publication and patent applications are fully compatible with the methods and systems described herein to measure an analyte. In some embodiments, near-infrared, fluorescence, or both types of spectral data may be used in addition to or place of Raman spectral data. The SD to mean ratio of the intensity values over the unique patterned region of the spectrum, 300-1700 cm⁻¹, ranges from 0.03 to 0.1. The tissue spectral signatures can be attributed to the presence of Raman-active components (such as blood analytes, collagen I and III, structural proteins in the epidermis and dermis, and sub-cutaneous lipids) and endogenous fluorophores. While the use of excitation light in the near-infrared (NIR) considerably reduces levels of autofluorescence, the presence of a broad background can still be observed in the acquired spectra. The strongest Raman peak is observed at ca. 1445 cm⁻¹ and other prominent features are located at approximately 859, 938, 1004, 1273, 1302 and 1655 cm⁻¹, which is consistent with prior in vivo tissue observations. Expectedly, the Raman bands of glucose are masked in the myriad signals of other constituents and cannot be uniquely assigned by visual inspection alone. This necessitates the use of multivariate algorithms to identify subtle changes and to link such changes to the glucose concentrations at different time points.

In accordance with various embodiments, the present approach can be used to predict glucose concentrations with only the first reference concentration from each subject being used to develop the model. To compare the efficacy of the current method to more established approaches, PLS calibration was also used to estimate the glucose concentrations based on the acquired Raman spectra. Because the PLS calibration (or any other analogous implicit calibration technique such as PCR or SVR) method requires significantly more reference concentrations to build a model, a cross-validation procedure is implemented to test the predictive power of the model. While the leave-one-out cross-validation routine (LOOCV; described in greater detail below) avoids some of the pitfalls encountered in auto-prediction, alternative methods such as PLS, PCR, or SVR may yet result in an apparently functional model (due to “overtraining”) that cannot actually be used for prospective prediction. Despite this possibility, the PLS LOOCV procedure provides a method for comparison while also highlighting the need for the approach as described by the present invention.

To illustrate the conventional approach and allow comparison to the approach of exemplary methods of the present invention, PLS models were created based on the number of loading vectors that provide the least error in cross-validation. Models such as the PLS model are discussed in U.S. Pat. Nos. 8,355,767 and 9,103,793, the entire contents of both patents being incorporated herein by reference. The PLS models did not explicitly address the physiological dynamics issue. Here, LOOCV approach was used to provide concentration estimates, because of the limited number of data points available per individual. In LOOCV, the data from a particular time point is eliminated, and the PLS model developed on all the other points is used to predict the concentration at that time point optimizing agreement with the reference measurement.

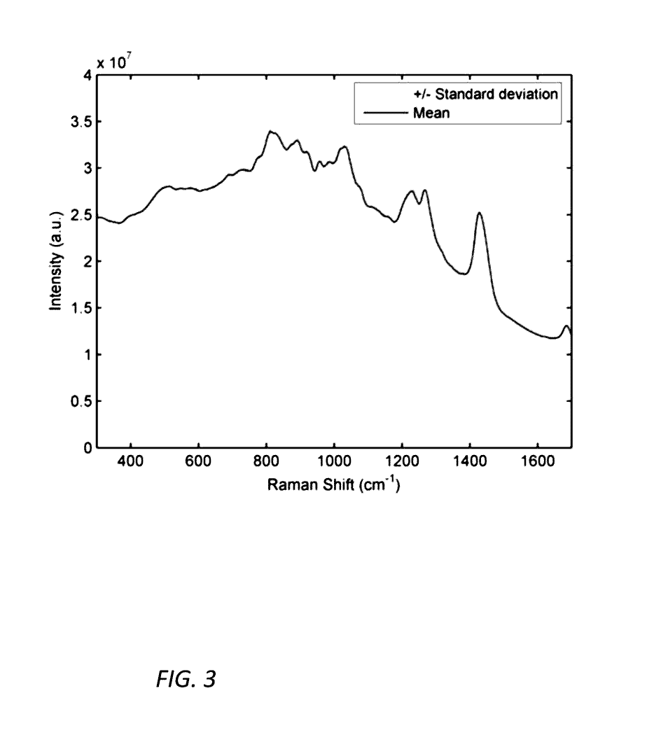

FIG. 4 displays the results of prediction according to exemplary methods of the present invention (red diamond) and PLS LOOCV (black circle) in a representative subject where the blue squares depict the measured blood glucose values during an OGTT. Blood was drawn every 10 min and analyzed using a clinical glucose system (HemoCue, Inc., Brea, Calif.) to evaluate the subject's response and obtain the measured blood glucose values. The measured concentrations show the expected rise in glucose levels due to ingestion of a sugar-rich drink followed by subsequent recovery to (nearly) euglycemic levels owing to the normal insulin response. The recovery would be delayed or absent if a diabetic subject were tested. Note that the present model (root mean squared error of prediction, RMSEP=5.14 mg/dL) exhibits significantly better prediction accuracy in comparison with the PLS LOOCV estimation (root mean squared error of cross validation, RMSECV=13.64 mg/dL). The better estimation using the present system can be attributed to two factors: suitable correction for the physiological lag between blood and ISF glucose and isolation of the baseline shifts and system drifts. The effect of the former can be viewed in the initial 60 minutes when the glucose levels of the subject rise sharply. During this time frame, the system predictions match considerably better with the reference concentrations than the PLS estimates by appropriately modeling the transient discrepancies. Conventional calibration methods exhibit systematic errors during rapid excursions even in the presence of a positive correlation between blood and ISF glucose.

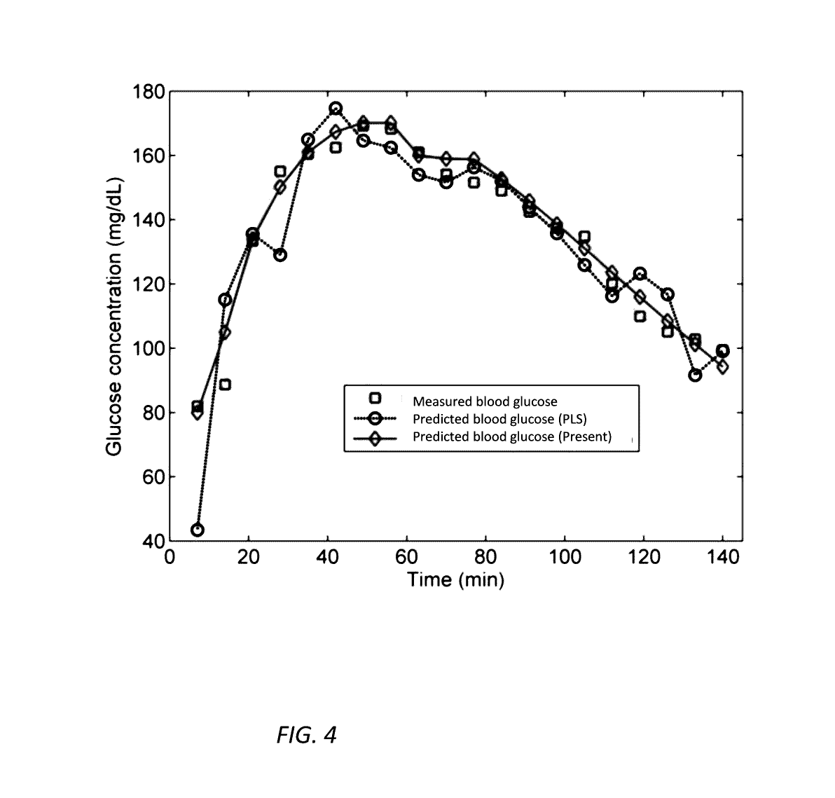

To better illustrate the predictive power when multiple human subject data sets are included in the analysis, the results of the present model are plotted on the Clarke error grid (FIG. 5), a widely used method for quantifying the clinical usefulness of glucose predictions. Predictions in zones A and B are regarded as acceptable, and predictions in zones C, D, and E are considered to be potentially dangerous if used for clinical management. The RMSEP and the R² value (coefficient of determination) are computed to be 0.54 mM (1 mM of glucose=18 mg/dL) and 0.97, respectively. Critically, all the glucose predictions over the entire human subject dataset reside in the clinically acceptable regions—even when the glucose levels are relatively low (4-6 mM). This result is of great value as a key benefit of a continuous glucose monitoring system in the real-time detection of hypoglycemic states. A common motif in diabetes care is the lack of immediate knowledge regarding low blood glucose excursions in over-medicated patients resulting in serious consequences including diabetic coma. Embodiments of the present invention address this need by non-invasively and, optionally, continuously estimating blood glucose trends that are predictive of both hypoglycemic and hyperglycemic blood glucose excursions.

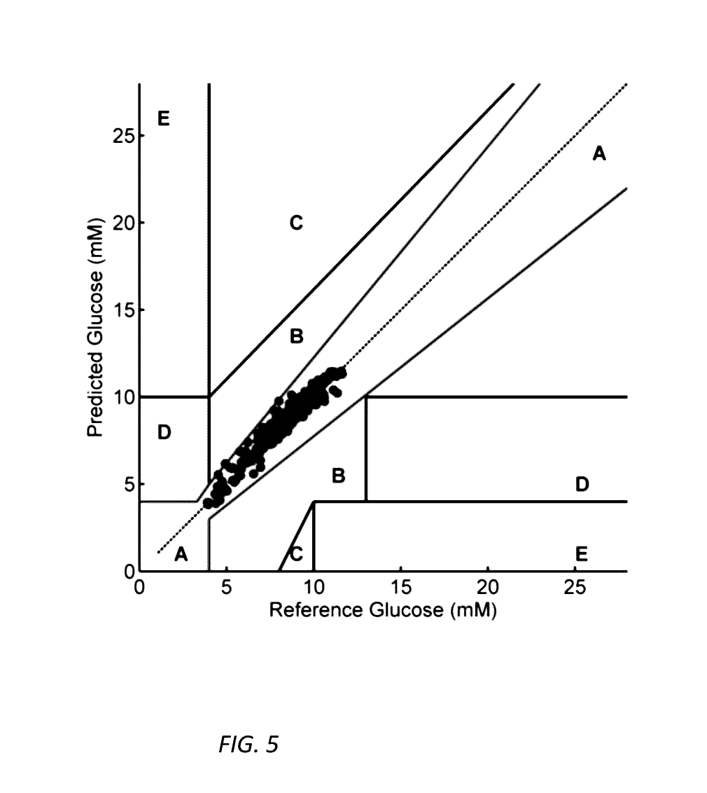

FIG. 6 presents a table that summarizes the results of the present model predictions as well as the corresponding PLS LOOCV estimates. The reduction in error on application of the present model, when compared to the PLS model estimates, ranges from nearly 18% to 59% with an average value of 35.5% computed over the 8 subjects.

The glucose diffusion process can be characterized using a two-parameter model (k₁, k₂). This formulation ensures that the rate of glucose uptake by the subcutaneous tissue is also addressed in the mass diffusion process. In each of the volunteers, k₁ had a larger numerical value in relation to k₂ signifying that the blood glucose rise was faster than the return to euglycemic levels. This is consistent with typical observations in glucose tolerance studies where the increase in blood glucose levels following ingestion of glucose solution is rapid in relation to the subsequent insulin-mediated glucose clearance from the blood (by the cells) and, thus, the corresponding return to normal blood glucose levels. Subjects with impaired glucose tolerance will tend to exhibit significant changes in the determined rate constants, especially k₂.

This formulation can model situations where, during the time of decreasing glucose levels, ISF glucose may fall in advance of blood glucose and reach nadir values that are lower than the corresponding blood glucose levels. It has been reported that ISF glucose levels can remain below blood glucose concentrations for fairly long periods of time following correction of insulin-induced hypoglycemia. Such findings can be understood by reference to the so-called push-pull phenomenon according to which the glucose is pushed from the blood to the ISF compartment during the rising phases and the glucose is recruited from the ISF to the surrounding cells during the falling phases. If such a model is accurate, preferred embodiments of the methods herein can re-calibrate by adjusting the corresponding k₂ value.

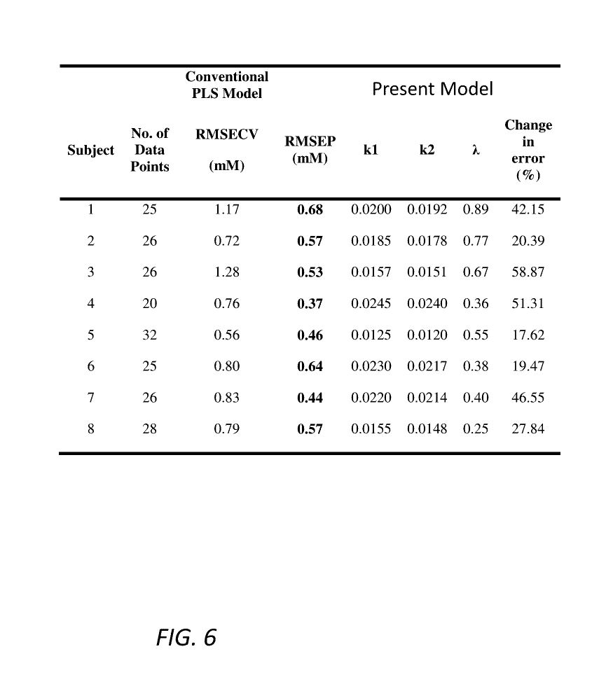

To further validate systems and methods of the present invention, a demonstration of the causality of the glucose concentration to the acquired spectral information can be utilized especially as the intrinsic glucose signal is significantly smaller than that of several other blood-tissue matrix constituents. Moreover, time-dependent physiological processes or variations specific to an instrument that happen to be correlated with the glucose levels have often been found to dominate classical implicit calibration models, especially for non-specific measurement modalities. To investigate the robustness of concentration estimates to chance correlations (spurious factors), an F-test was used to compare the squared error of prediction (SEP) to the standard deviation of the glucose concentrations within the prediction data set (SDP) and, therefore, to assess if the variability of the predicted concentrations is greater than might be expected by chance. Using the values listed in the table shown in FIG. 7, the F-values for the PLS LOOCV estimates and estimates according to the present methods were calculated to be 4.99 and 13.28, respectively. For the PLS computation, the SEP was replaced with SECV. Clearly, both sets of F-values are statistically significant. Thus, the null hypothesis that the variance of errors of glucose predictions is the same as the variance of the reference glucose concentrations can be rejected. The table in FIG. 7 also lists the results for linear regression analysis of the prediction points for the PLS and kinetic model cases. The y-intercept for the kinetic model predictions is lower, the slope is closer to unity, and the R² value is higher. These results strongly indicate that the current glucose predictions are based on the spectroscopic properties of glucose rather than on chance variations or correlative response between glucose and other matrix constituents. Notably, since the present models do not use the standard input of an array of reference concentrations, the possibility of building an apparently functional model based on incidental correlations is largely eschewed.

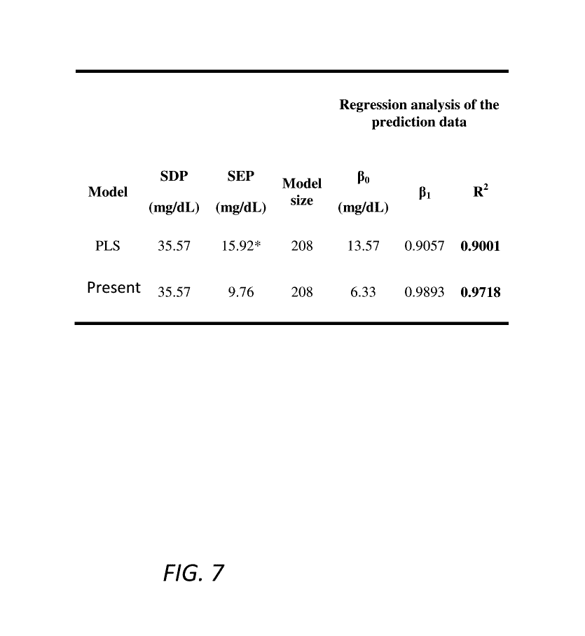

Difference plot analysis was also performed (FIG. 8) for further comparison of the methods. The Bland-Altman plots of FIG. 8 enable the investigation of the presence of any systematic difference between the reference and Raman measurements and to identify possible outliers. Here, the mean difference is the estimated bias and is found to be 0.5 mg/dL and 24.73 mg/dL for the PLS and system estimates, respectively. The corresponding 2SD limits are determined to be 31.9 mg/dL and 17.1 mg/dL, respectively. As per ISO 15197 guidelines, these 2SD limits should be less than 15 mg/dL for glucose concentration below 75 mg/dL and should be lower than 20% for any value higher than 75 mg/dL. These findings therefore suggest that the combination of the present approach and Raman spectroscopy provide clinically viable predictions, especially in terms of single-individual prediction.

With regard to glucose monitoring, the present model as described herein can predict impending hypo- and hyperglycemic excursions to allow a diabetic patient to take necessary corrective action. Further, embodiments of the present invention can be employed to study physiological changes (for example, in micro- and macro-vasculature) due to the onset of diabetes through their ability to characterize the glucose transport process in the circulation system and the ISF. While the present day standard of care primarily involves interpretation of changes in blood glucose, measurement of ISF glucose levels may be more important for specific clinical conditions such as the persistence of impaired cognition for prolonged periods of time after correction of hypoglycemia.

The present invention takes advantage of the potential of a spectroscopic method for tracking bioanalytes in a dynamic system with minimal a priori concentration information. The present approach is able to make accurate predictions in clinical datasets acquired from human subjects in the presence of myriad non-analyte specific variations. The performance metrics of the present algorithm exceed that of the conventional PLS calibration method, which is attributable to the twin advantages of accounting for the physiological lag between blood and ISF glucose and avoiding the baseline shifts and system drifts. The present formulation can be readily extended to quantify analytes using other spectroscopic signatures such as infrared absorption and thermal emission that offer higher sensitivity in comparison to Raman spectroscopy measurements.

Given the inherent non-invasive nature of vibrational spectroscopy, the combined method is appropriate as a real-time clinical adjunct for continuous monitoring of glucose and other blood analytes, e.g., creatinine, urea and bilirubin, in critical care patients and in neonates where frequent blood withdrawal is particularly problematic. Application of this minimally perturbative approach can lay the foundation in the near future for a novel spectroscopic assay for glucose tolerance testing requiring no blood withdrawal. The scope this method extends beyond in vivo diagnostics to microfluidics investigations as well as recalcitrant industrial process monitoring where intermediate sampling of the specimen would compromise its identity.

In the present approach, SVD is performed on the recorded time-resolved spectral data matrix, Y, to effectively extract the critical time trace information of the analyte(s) of interest. SVD is used to decompose the data into orthonormal abstract spectra (represented by the V^{T} matrix) and time-trace of the concentration information (represented by the U matrix). Σ is the classical diagonal matrix, where its elements are the singular values of Y. For noiseless data, the number of system constituents, exhibiting detectible spectral signatures, can be obtained from nₛ, the number of non-null elements in Σ. However, for spectral data acquired under routine experimental conditions, the singular values will not be zero beyond the expected value of nₛ but will tend to decay in an approximately exponential manner.

Experimental time-resolved Raman spectra contain noise contributions that are both homoscedastic (where the noise is independent of the signal intensity) and heteroscedastic (where the noise is dependent on the signal intensity). The former is primarily attributable to detector noise, laser intensity fluctuations and contributions from the background. The primary source of the latter is the shot noise component, which occurs when the number of collected photons is small and hence a fluctuation in the number of detected photons causes a detectable change in the measured spectrum. The standard deviation of shot noise scales as the square root of the signal intensity. Specifically, Raman spectra with longer excitation wavelengths exhibit noise with a more homoscedastic character, since longer excitation wavelengths require higher sensitivity detectors with higher noise contributions. Unlike shot noise, the singular value associated with baseline fluctuations can be considerably higher, leading to an overestimation of nₛ.

To minimize this adverse impact of baseline fluctuations, first derivative-based preprocessing of the spectral dataset is pursued. The application of the first derivative (of the spectral intensity with respect to Raman shift) completely cancels offset fluctuations and effectively minimizes more complex baselines fluctuations. Care is also taken to account for the expected noise dependence on wavenumber and time. Therefore, for our analysis, SVD is actually performed on:

Y_{w}′=diag(wᵥ)×der[Y]×diag(wₜ) (11)

where wₜ and wᵥ are the inverse of the expected noise standard deviation as a function of the wavenumber and time, diag( ) is a diagonal matrix with diagonal elements given by the vector in the brackets, and der[ ] performs the first derivative on the corresponding matrix columns. The first derivative was performed in the Fourier domain as described in the literature, without apodization or phase correction. As a consequence, the output of SVD becomes: Y_{w}′=U_{w}×Σ× V_{w}^{T}′. The normal U and V matrices are recovered by the following transformations: U=U_{w}×(diag(wₜ))⁻¹ and V^{T}=int[diag(wᵥ))⁻¹×V_{w}^{T}], where int[ ] performs the integral on the corresponding matrix columns. This process effectively pushes singular values associated with baseline fluctuations below those for genuine signals.

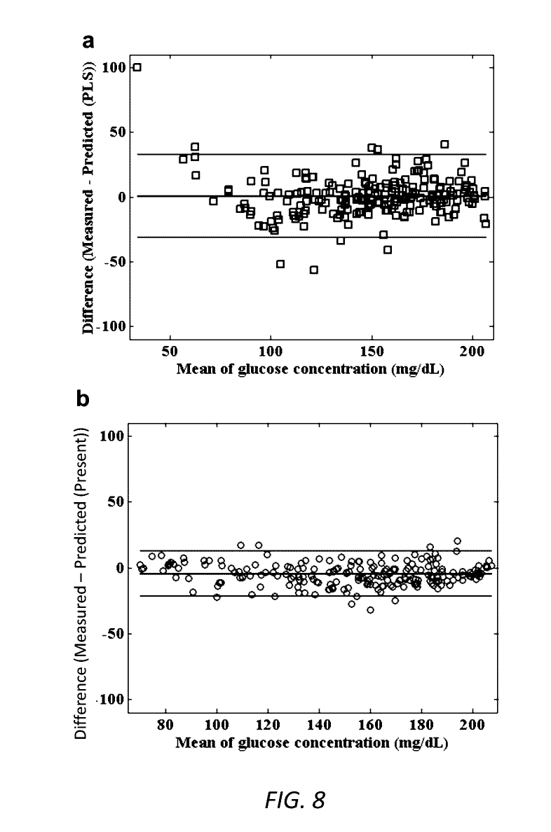

FIG. 9 depicts a method 900 for non-invasively measuring blood glucose concentration according to various embodiments. In step 911, a reference glucose concentration value is obtained from a blood sample of a patient. In step 913, first spectral data is obtained through a tissue layer of the patient. In step 915, the first spectral data is calibrated using the reference glucose concentration value to generate calibrated spectral data.

In step 917 of the method 900, a kinetic model parameter shift that reduces a residual between a glucose concentration profile computed from kinetic model parameters and a glucose concentration profile obtained from the calibrated spectral data is iteratively determined to provide final kinetic model parameters. In step 919, a glucose concentration value obtained from second spectral data measured through the tissue layer of the patient after a time interval is transformed using the final kinetic model parameters to generate a calibrated glucose concentration value.

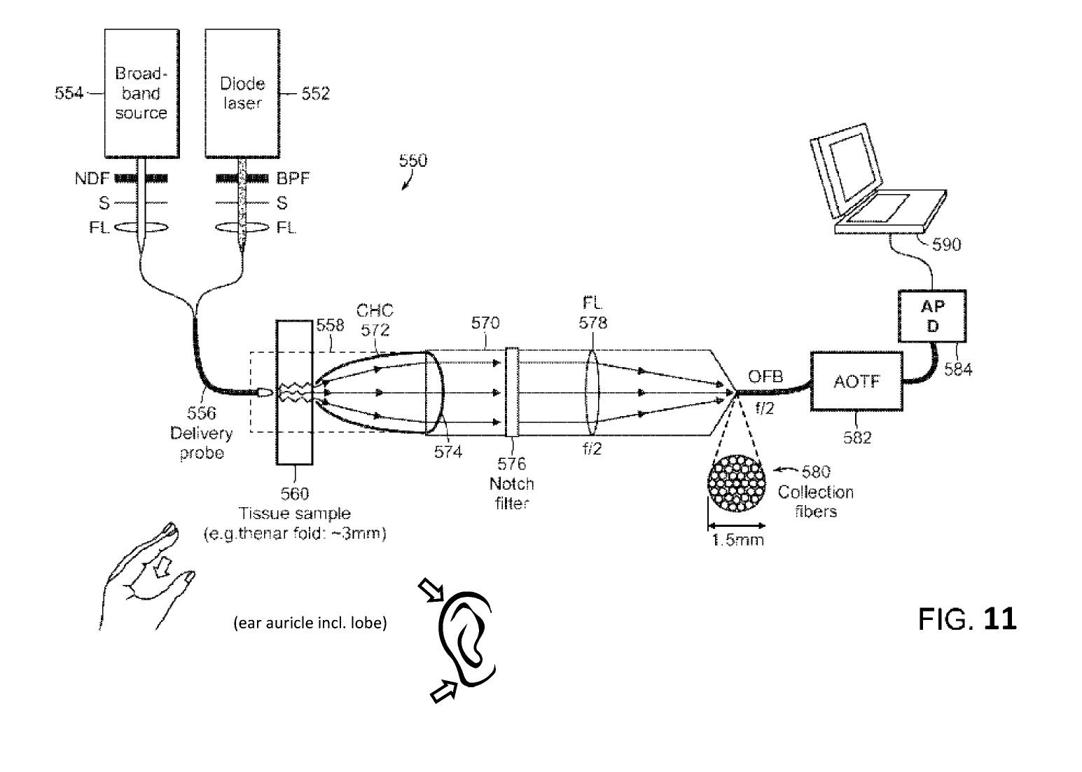

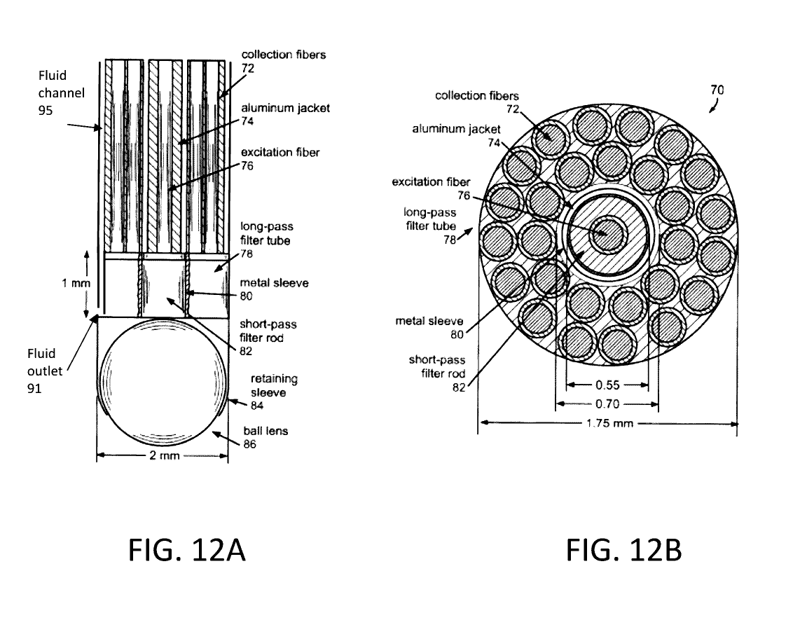

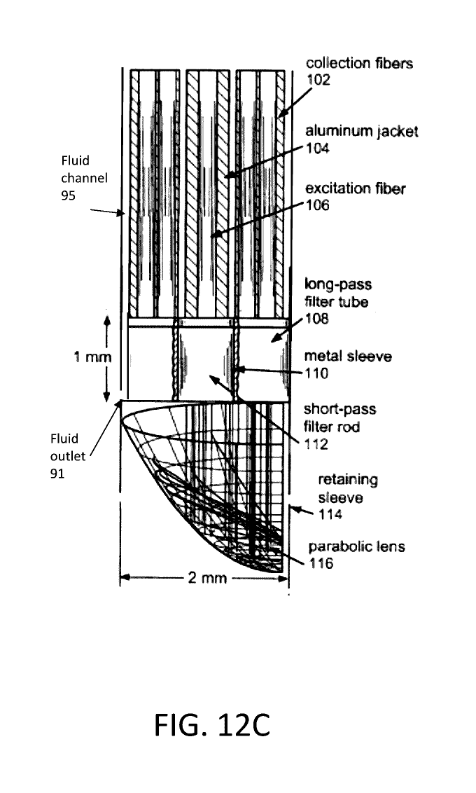

In step 911, a reference glucose concentration value can be obtained from a patient in a number of ways including, but not limited to, withdrawing blood using standard phlebotomy techniques and using a clinical glucose monitor. Non-limiting examples of clinical glucose monitors are the HemoCue® family (HemoCue America, Brea, Calif.). In step 913, first spectral data can be obtained through a tissue layer of a patient using, as only one example of many, a spectroscopic probe. The spectroscopic probe can use, for example, Raman or near-infrared spectroscopy and can contain light fibers as described in greater detail below with reference to FIGS. 12-15.

In step 915, the first spectral data can be calibrated using the reference glucose concentration value to generate calibrated spectral data by, for example but not limited to, noting the absolute values of and relationships between specific spectral features at a specific calibrated glucose concentration obtained from the biological calibration sample as described in the foregoing framework and in particular with reference to step 14 of method 20 depicted in FIG. 1B.

The measurement of the calibration sample can be used for a period of hours, days, or weeks depending on the condition of a particular patient. For routine monitoring of the glucose level of an otherwise healthy patient, the measurement of the calibration sample can extend for at least 24 hours or more.

In step 917, a kinetic model parameter shift that reduces a residual between a glucose concentration profile computed from kinetic model parameters and a glucose concentration profile obtained from the calibrated spectral data is iteratively determined to provide final kinetic model parameters by, for example but not limited to, employing portions of the above-described framework and, in particular, with reference steps 16, 18, 22, 24, 26, and 27 of method 20 depicted in FIG. 1B.

In step 919, transformation of a glucose concentration value obtained from second spectral data measured through the tissue layer of the patient after a time interval using the final kinetic model parameters to generate a calibrated glucose concentration value may be performed, for example but is not limited to, using portions of the framework described above and in particular with reference to step 28 of method 20 depicted in FIG. 1B.

Systems and devices for use with the measurement calibration methods of the present invention can include a variety of forms. For example, a spectroscopy system may be used to illuminate a tissue and receive radiation scattered from the tissue. The system may filter the excitation or emission light to restrict the range of wavelengths. One or more imaging modalities or vibrational spectroscopic techniques may be used including Raman or infrared spectroscopy or diffuse reflectance. The radiation may be detected after back-scattering from the tissue (i.e., a reflection geometry) or after scattering through the tissue (i.e., a transmission geometry).

A preferred embodiment of a system 420 for measuring an analyte can include a tunable source 422, a fiber optic probe 426 and a detector system 424 as shown in FIG. 10. The distal end of the probe 426 includes a central Raman delivery fiber 440, an optional diffuse reflectance delivery fiber 442, surrounding collection fibers 444, a short pass filter rod 446 that filters the Raman excitation light, a notch filter tube 448 that filters the light being collected and an optical concentrator (such as a compound parabolic concentrator or CPC) 450 for coupling light onto the tissue 460 and providing improved light collection efficiency.

Utilizing multiple lasers with a single band pass filter and photodetector can be effective to miniaturize the device. In this embodiment, the excitation system 422 uses several laser diodes emitting light at different respective wavelengths. Several laser diodes excite different spectral regions and employ only a single photodetector in the collection system 424 to receive spectral information. These spectral regions can be selected using wavelength selection methods. In embodiments that use Raman spectroscopy, the excitation source 422 can include an 830 nm diode laser. The average intensity of the source may be 300 mW in a ˜1 mm² spot. In accordance with various embodiments, the collection system 424 can include an f/1.8 spectrograph coupled to a liquid nitrogen-cooled CCD (1340×1300 pixels) to detect the emitted spectroscopic signals.

Coupled with a small diode laser, a fiber optic probe for excitation and collection, and a detector array, a hand-held Raman unit can have extensive applications in the field of disease diagnostics, analyte detection, and analytical and bio-analytical chemistry. For example, a miniaturized Raman system that utilizes the calibration and measurement methods discussed herein can be employed as a non-invasive continuous glucose monitor. The device can be worn around the wrist or around the waist, as is the case for electrochemical minimally invasive sensors. Another potential application is the use of a miniaturized Raman system for testing withdrawn blood samples for glucose levels. Further descriptions of systems that may complement or be used in conjunction with the present invention may be found in U.S. patent application Ser. No. 13/167,445, filed Jun. 23, 2011, the entire contents of which is incorporated herein by reference.

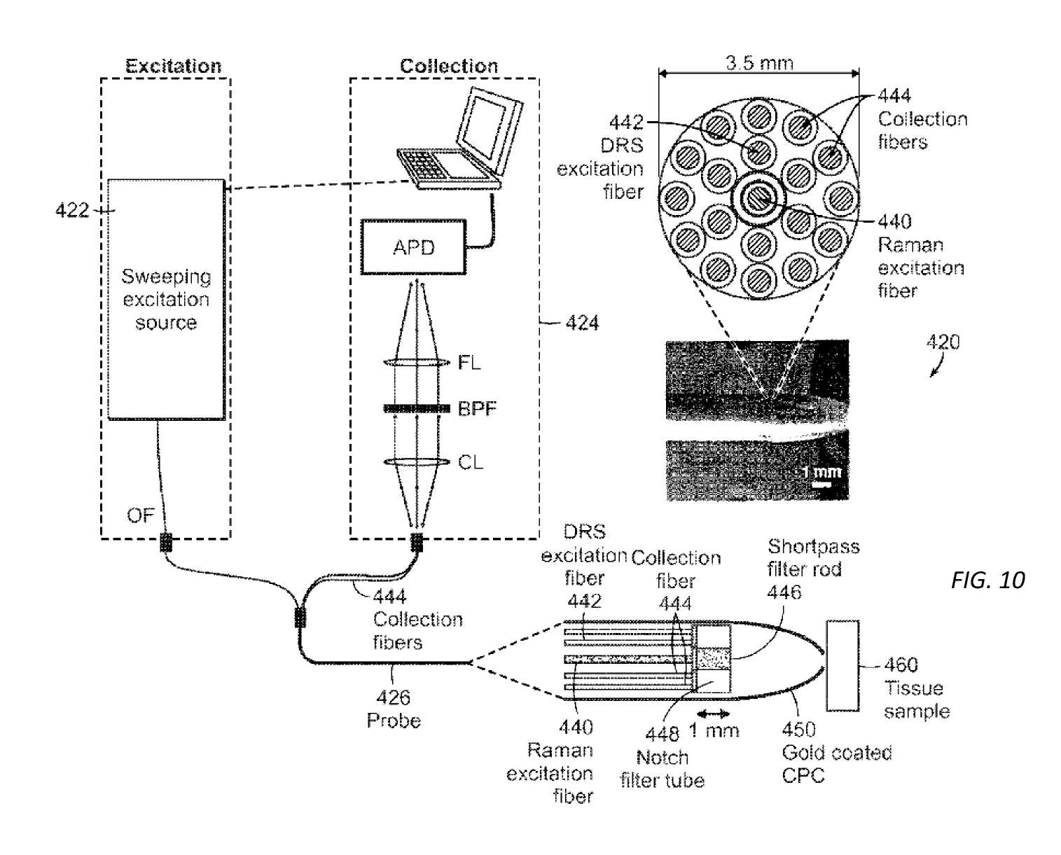

FIG. 11 illustrates a system using a compound hyperbolic concentrator. A compound hyperbolic concentrator (CHC) is a non-imaging optical concentrator that efficiently collects the light emanating from highly scattering biological tissues while using optical systems with very low numerical apertures (NA). Unlike a compound parabolic concentrator (CPC), a CHC allows for the collimation of scattered light directions to within an extremely small range of angles while also maintaining practical physical dimensions. Such a design allows for the development of a very efficient and compact spectroscopy system for analyzing highly scattering biological tissues.

Many optical systems can receive light at only a limited range of angles specified by their numerical apertures. In addition, efficient light collection is crucial for a tissue Raman spectroscopy system due to the inherently weak nature of Raman scattered light. For this reason, spectroscopic instruments used for the analysis of biological tissues often require a light collection device that redirects the scattered light entering at steep angles to be within a limited range of angles with respect to the receiving optical system. This had been previously achieved with great efficiency by the use of a CPC. However, the CPC length becomes impractically long if a low numerical aperture is desired. For example, certain dielectric coatings on optical filters are effective only at a very limited range of incident angles. The present embodiment of the invention uses a CHC to efficiently collimate light scattered at wide angles from biological tissues into a very low numerical aperture while maintaining practical physical dimensions.

The CHC reflector surface is defined by rotating the curve formed by two tilted and offset hyperbolas with the input aperture defined by their foci. It is analogous to the CPC where the curve is similarly formed by two parabolas. However, in addition to the reflector, the CHC 572 is also coupled with a matching focusing lens 574 with the focal length defined by the distance between the output aperture and the focus on the opposite side of the symmetric axis of the hyperbola. This combination effectively causes the CHC to function as a CPC with a much longer longitudinal dimension. The CHC surface is polished to optical grade (0.3 micron) finish and coated with the appropriate reflective material optimized for the desired wavelength. The focusing lens is coated with an antireflective coating appropriate for the applicable wavelength. Using computer-controlled machining and electroforming techniques, a 0.06 NA CHC with a 4 mm diameter input aperture was fabricated at a length of about 12 cm. (A CPC with the same NA and input aperture would require the length to be 4 times longer.) The incident light entering the CHC at a steep angle (up to +/−90 degrees to normal) exited the CHC at angles within +/−3.4 degrees to normal. With appropriate modifications in the design, an even smaller range of angles can be achieved. The collimated light can then be filtered, focused and collected with ease, even in optical systems with a very limited range of acceptable angles. This CHC was used to collect Raman spectra from human skin on a portable transmission-mode Raman spectroscopy system 550 as shown in FIG. 11. Besides collection of Raman spectra from skin tissues in transmission mode, the proposed CHC design can have many other uses in spectroscopy systems requiring efficient collimation of nearly isotropically scattered light as, for example, from biological tissues.

The system 550 can include a diode laser 552, an optional broadband source 554, along with suitable filters (BPF, NDF), shutters (S), and lenses (FL) to couple excitation light into a delivery probe. The probe 556 can be aligned with a light collection system 570 using a housing 558. This system can be used, for example, to transmit light through tissue 560, such as the thenar fold of the human hand. A CHC 572 within the housing 570 collects transmitted light and, with lens 574, filter 576 and lens 578, couples the light into a collection fiber bundle 580. An acousto-optic tunable filter (AOTF) 582 can be used to select wavelengths of light to be detected by detector 584. A system processor and controller 590 can be used to process spectral data and operate the system as described herein.

In accordance with various embodiments, the system processor and controller 590 can contain a memory and a processor. The memory may contain processor-executable instructions that, when executed by the processor, allow the system processor and controller 590 to perform steps of the measurement calibration method described above. The processor-executable instructions may also be provided on a non-transitory computer readable medium in accordance with an embodiment of the present invention.

FIGS. 12A-12C show longitudinal and transverse views of preferred embodiments of a spectroscopic probe for measuring an analyte. These embodiments can be inserted within needle probes or the distal ends thereof can be configured with a needle-tip shape. The apparatus 70 can include a two-piece, multiple (for example, dual) wavelength micro-optical dielectric filter module for minimizing and preferably eliminating fiber Raman background in the delivery and collection fibers. This module consists of a rod 82 carrying the excitation dielectric filter coating on one plane face, fitted into the tube 78 carrying the collection dielectric coating on one plane face of the tube. Rods and tubes of the embodiment may be made of either sapphire or fused silica and are separately coated with their respective filters prior to assembly. The rod is wrapped or coated with a thin sheet of metal 80 to provide optical isolation between the components. The module is then placed at the distal end of the probe between the fiber bundles and a lens system for collimating the light beams having a lens 86 such as, for example, a ball lens. The lens collects light from high angles and a large area effectively overlapping excitation and collection regions. The ball lens can be fabricated and supplied by Edmund Industrial Optics, New Jersey. In a preferred embodiment, sapphire lenses that are coated with anti-reflection coatings and having an appropriate index for angular acceptance, for example, 1.77 are fabricated by MK Photonics, Albuquerque, N. Mex. Although it is expensive to obtain high quality interference filters at this scale, the cost of the filters is independent of the number of pieces coated; thus, it is possible to coat many filters at once thereby reducing the construction cost of each probe. Furthermore, through additional coating runs, the filter size can be adjusted to create smaller diameter probes for various applications. In a preferred embodiment, the filters are deposited on sapphire or quartz rods and tubes for proper registration with fibers. According to various embodiments, the apparatus may include a fluid channel 95 that can carry fluid between the proximal end and the distal end of the apparatus. The fluid may enter or exit the apparatus through the fluid outlet 91. Additional systems, devices, and probes that may be used in conjunction or as part of the present invention may be found in U.S. Pat. No. 7,647,092, U.S. patent application Ser. No. 10/407,923 filed on Apr. 4, 2003, U.S. patent application Ser. No. 13/167,445 filed on Jun. 23, 2011, and International Patent Application No. PCT/US2011/046750 filed on Aug. 5, 2011, the entire contents of said patent and patent applications being incorporated herein by reference.

FIG. 12C shows a longitudinal view of an alternate preferred embodiment having a paraboloidal mirror disposed in the lens system. The collection angle can be in the range of 0 to approximately 55° with a collection diameter of approximately 1 mm. The paraboloidal mirror collects light from a wider angle and a larger area. According to various embodiments, the apparatus may include a fluid channel 95 that can carry fluid between the proximal end and the distal end of the apparatus. The fluid may enter or exit the apparatus through the fluid outlet 91.

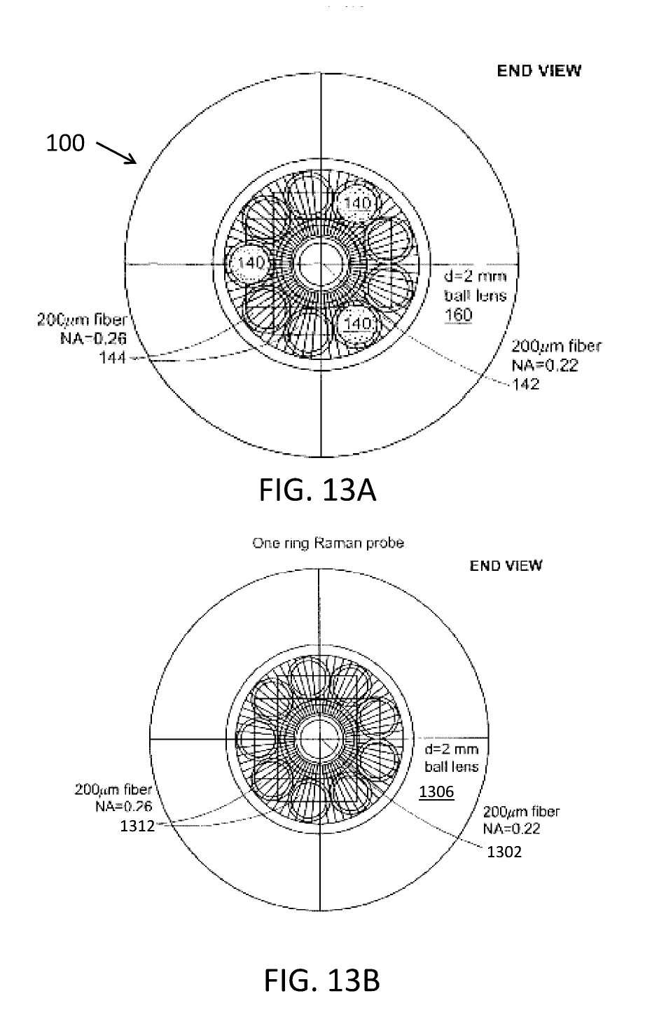

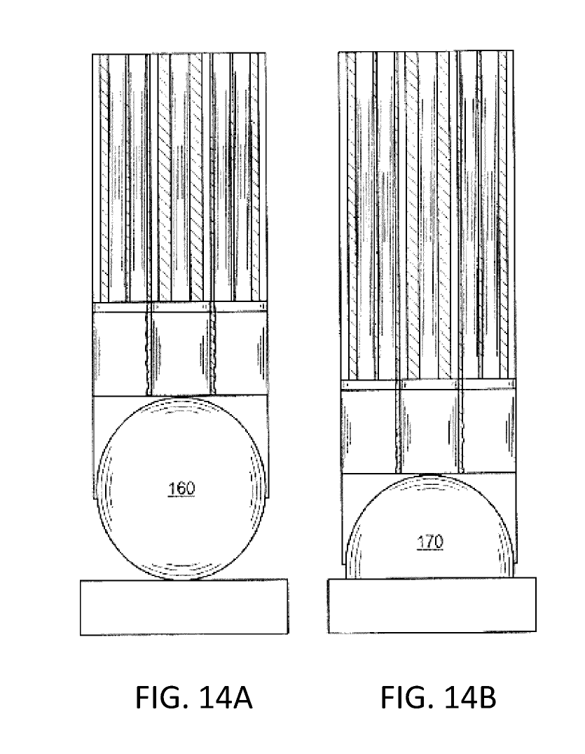

FIG. 13A illustrates an end view of a design in which a first group of 3 collection fibers 140 are used to collect reflected light and 3 pairs of fibers 144 collect the Raman light passing through ball lens 160. An exemplary embodiment comprises a reduced diameter 9-around-1 probe 100 and excitation/collection trajectories through a ball lens 160 that contacts tissue. The filter module has a filter rod placed on the delivery fiber with transmittance from 300-830 nm and no transmittance (<1%) beyond 850 nm. A filter tube placed on the collection fibers has transmittance from 300-810 nm and from 850-1000 nm and with a narrow 40 nm band centered at 830 nm having low transmittance. An end view of the probe is shown in FIG. 13A with collection fibers 144 positioned in a circular array around central excitation fiber 142. The central fiber 142 directs light through the forward looking probe with lens 160 in FIG. 14A or the half-ball lens 170 of FIG. 14B. In an alternative embodiment shown in FIG. 13B, light is delivered via the central excitation fiber 1306 through the ball lens 1306 and collected through the ball lens 1306 using collection fibers 1512 that are all similarly adapted or filtered to collect one type of light, e.g., Raman, near infrared, or diffuse reflectance light.

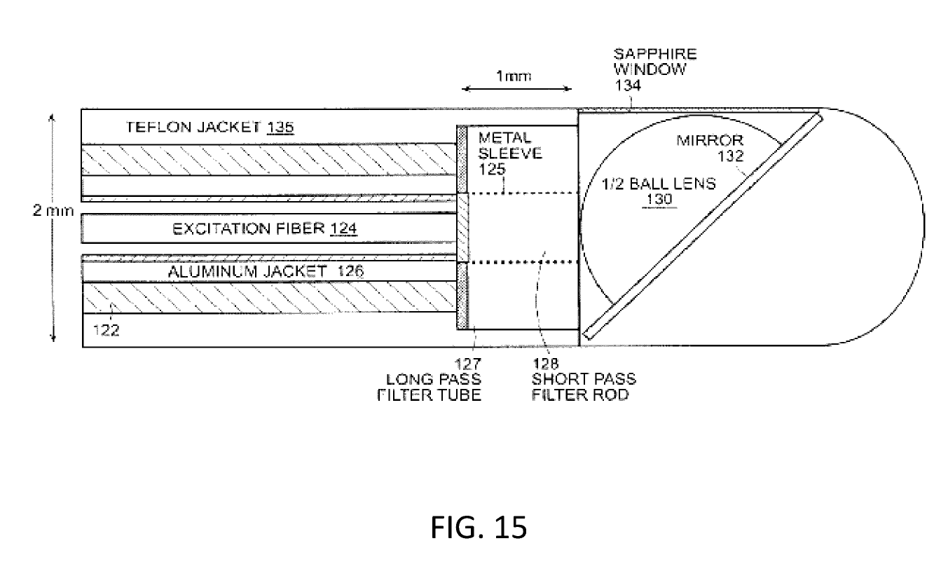

A side looking probe 120 is shown in FIG. 15 in which a half ball lens 130 is in contact with a mirror 132 to reflect light from excitation fiber 124 and filter rod 128 through sapphire window 134. Light returning from the tissue is reflected into collection fibers 122 through long pass filter tube 127. A metal sleeve 125 surrounds filter 128. An aluminum jacket surrounds the excitation fiber 126. A Teflon jacket 135 provides the cylindrical tube that forms the outer wall of the probe.

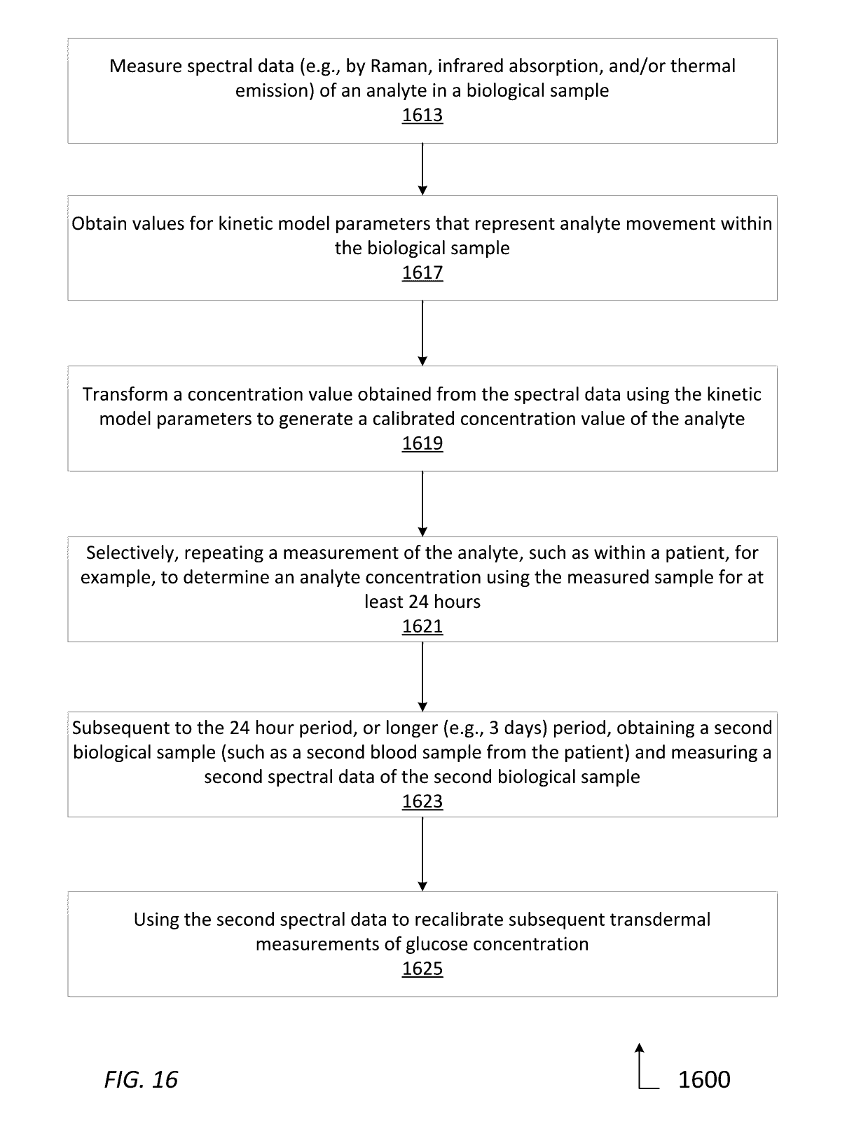

FIG. 16 depicts a method 1600 of measuring an analyte according to various embodiments. The method includes measuring spectral data of an analyte in a biological sample (step 1613). The method also includes obtaining values for kinetic model parameters that represent analyte movement within the biological sample (step 1617). The method also includes transforming a concentration value obtained from the spectral data using the kinetic model parameters to generate a calibrated concentration value of the analyte (step 1619).

Step 1613 of measuring spectral data of an analyte in a biological sample may include, but is not limited to, acquiring Raman spectral data including data from glucose with a spectroscopic instrument through a tissue of a patient as described above. Step 1617 of obtaining values for kinetic model parameters that represent analyte movement with the biological sample may include, but is not limited to, utilizing steps of the methods 10, 20 described above with reference to FIGS. 1A and 1B. Alternatively, step 1617 can include accessing a memory or database containing kinetic model parameters.

Step 1619 of transforming a concentration value obtained from the spectral data using the kinetic model parameters to generate a calibrated concentration value of the analyte can include, but is not limited to, computing the dot product of a regression matrix calculated with final kinetic model parameters and a concentration profile computed from the spectral data to generate a calibrated concentration value as described in the framework above and with reference to FIG. 1B. Step 1621 of selectively repeating a measurement of the analyte to determine an analyte concentration using the measured sample for at least 24 hours can be performed may include, but is not limited to, the measurement steps as described above with reference to Step 1613 and above. As described above, the measured calibration sample can be used to calibrate measurements for at least 24 hours in some embodiments. In some embodiments, the step can include transforming concentration values obtained from the selectively measured spectral data using the additional kinetic model parameters to generate calibrated concentration values of the analyte for at least 24 hours.

Step 1623 of obtaining a second biological sample subsequent to a 24 hour or longer period and measuring a second spectral data of the second biological sample can include, for example, drawing a second blood sample from the patient and acquiring Raman or other spectral data including data from glucose with a spectroscopic instrument as described above. Step 1625 of using the second spectral data to recalibrate subsequent transdermal measurements of glucose concentrations can include using the second spectral data (which may be calibrated using the second biological sample) to calibrate further analyte measurements.

While the present inventive concepts have been described with reference to particular embodiments, those of ordinary skill in the art will appreciate that various substitutions and/or other alterations may be made to the embodiments without departing from the spirit of the present inventive concepts. Accordingly, the foregoing description is meant to be exemplary and does not limit the scope of the present inventive concepts

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

1. A method of measuring an analyte comprising:
obtaining a biological calibration sample;
measuring first spectral data of an analyte in a biological sample;
calibrating the first spectral data using measured data from the biological calibration sample to generate calibrated spectral data;
iteratively determining a kinetic model parameter shift that reduces a residual between a concentration profile computed from kinetic model parameters and a concentration profile obtained from the calibrated spectral data to provide selected kinetic model parameters; and
transforming a concentration value obtained from second spectral data measured after a time interval using the selected kinetic model parameters to generate a calibrated concentration value of the analyte.

2. The method of claim 1, wherein the analyte is glucose.

3. The method of claim 1, wherein the biological calibration sample comprises a sample of blood.

4. The method of claim 1, wherein the kinetic model parameters represent a mass-transfer model.

5. The method of claim 4, wherein the mass-transfer model comprises a two-compartment mass-transfer model that characterizes physiological lag between analyte concentrations in blood and interstitial fluid.

6. The method of claim 1, wherein obtaining the biological calibration sample includes using a clinical glucose meter to analyze a sample of blood.

7. The method of claim 1, wherein the first spectral data and second spectral data comprise one or more of Raman spectra, fluorescence spectra, or near-infrared spectra.

8. The method of claim 1, wherein calibrating the first spectral data includes first-derivative based preprocessing to minimize the impact of baseline fluctuations.

9. The method of claim 1, further comprising using a data processor for transforming a concentration value obtained from third spectral data measured after a time interval using the selected kinetic model parameters to generate a calibrated concentration value of the analyte.

10. The method of claim 1, wherein calibrating the first spectral data using measured data from the calibration sample to generate calibrated spectral data includes employing a least-squares method such as singular value decomposition, partial least-squares, or principal component regression to isolate relevant time-trace information in the first spectral data.

11. The method of claim 1, wherein iteratively determining a vector of kinetic model parameter shifts that reduces a residual between a concentration profile computed from kinetic model parameters and a concentration profile obtained from the calibrated spectral data to provide the selected kinetic model parameters includes integrating a differential equation representing a kinetic model to enable calculation of a computed concentration profile.

12. The method of claim 11, wherein iteratively determining a vector of kinetic model parameter shifts that reduces a residual between a concentration profile computed from kinetic model parameters and a concentration profile obtained from the calibrated spectral data to provide selected kinetic model parameters further includes calculating a residual matrix as a difference between the concentration profile computed from the kinetic model parameters and the concentration profile obtained from the calibrated spectral data.

13. The method of claim 12, wherein iteratively determining a vector of kinetic model parameter shifts that reduces a residual between a concentration profile computed from kinetic model parameters and a concentration profile obtained from the calibrated spectral data to provide the selected kinetic model parameters further includes calculating a Jacobian by unfolding the residual matrix into a long vector and calculating a forward finite-difference.

14. The method of claim 13, wherein iteratively determining a vector of kinetic model parameter shifts that reduces a residual between a concentration profile computed from kinetic model parameters and a concentration profile obtained from the calibrated spectral data to provide selected kinetic model parameters includes using the Jacobian to generate updated kinetic model parameters for the next iteration.

15. The method of claim 14, wherein iteratively determining a vector of kinetic model parameter shifts that reduces a residual between a concentration profile computed from kinetic model parameters and a concentration profile obtained from the calibrated spectral data to provide selected kinetic model parameters further includes calculating a new residual matrix using updated kinetic model parameters and comparing to the previous residual matrix to determine whether to continue iterating or whether to set the updated kinetic model parameters as the selected kinetic model parameters.

16. The method of claim 1, wherein transforming a concentration value obtained from second spectral data measured after a time interval using the selected kinetic model parameters to generate a calibrated concentration value of the analyte includes computing the dot product of a regression matrix calculated with selected kinetic model parameters and a concentration profile computed from second spectral data obtained after a time interval to generate a calibrated concentration value.

17. The method of claim 1, wherein the time interval is between 3 and 7 days.

18. The method of claim 1, wherein the time interval is between 1 and 3 days.

19. The method of claim 1, wherein the time interval is between 10 minutes and 24 hours.

20. A device for measuring calibrated glucose concentration values, comprising:
a probe including one or more light collection fibers and one or more light delivery fibers;
a light source coupled to the one or more light delivery fibers to deliver light to the skin of a patient;
a detector to receive light collected from the skin and output a plurality of spectral data; and
a computing device operatively coupled to the detector to receive the plurality of spectra, the computing device equipped with a processing unit and a memory to store processor-executable instructions wherein execution of the instructions causes the processing device to carry out a method for measuring an analyte comprising:
obtaining a calibration sample;
measuring first spectral data of an analyte in the sample;
calibrating the first spectral data using measured data from the biological calibration sample to generate calibrated spectral data;
iteratively determining a kinetic model parameter shift that reduces a residual between a concentration profile computed from kinetic model parameters and a concentration profile obtained from the calibrated spectral data to provide selected kinetic model parameters; and
transforming a concentration value obtained from second spectral data measured after a time interval using the selected kinetic model parameters to generate a calibrated concentration value of the analyte.

21. The device of claim 20, wherein the analyte is glucose.

22. The device of claim 20, wherein the first spectral data and second spectral data comprise one or more of Raman spectra, fluorescence spectra, or near-infrared spectra.

23. The device of claim 20, wherein the kinetic model parameters represent a mass-transfer model.

24. The device of claim 23, wherein the mass-transfer model comprises a two-compartment mass-transfer model that characterizes physiological lag between analyte concentrations in blood and interstitial fluid.

25. The device of claim 20, wherein the light source includes one of a diode laser or broadband source.

26. The device of claim 20, further comprising one or more optical filters located at the distal ends of the light delivery fibers or light collection fibers.

27. The device of claim 20, wherein calibrating the first spectral data includes first-derivative based preprocessing to minimize the impact of baseline fluctuations.

28. The device of claim 20, wherein the method performed by the processor further comprises transforming a concentration value obtained from third spectral data measured after a time interval using the final kinetic model parameters to generate a calibrated concentration value of the analyte.

29. The device of claim 20, wherein calibrating the first spectral data using measured data from the calibration sample to generate calibrated spectral data includes employing a least-squares method such as singular value decomposition, partial least-squares, or principal component regression to isolate relevant time-trace information in the first spectral data.

30. The device of claim 20, wherein iteratively determining a vector of kinetic model parameter shifts that reduces a residual between a concentration profile computed from kinetic model parameters and a concentration profile obtained from the calibrated spectral data to provide final kinetic model parameters includes integrating a differential equation representing a kinetic model to enable calculation of the concentration profile computed from the kinetic model parameters.

31. The device of claim 30, wherein iteratively determining a vector of kinetic model parameter shifts that reduces a residual between a concentration profile computed from kinetic model parameters and a concentration profile obtained from the calibrated spectral data to provide final kinetic model parameters further includes calculating a residual matrix as a difference between the concentration profile computed from the kinetic model parameters and the concentration profile obtained from the calibrated spectral data.

32. The device of claim 31, wherein iteratively determining a vector of kinetic model parameter shifts that reduces a residual between a concentration profile computed from kinetic model parameters and a concentration profile obtained from the calibrated spectral data to provide final kinetic model parameters further includes calculating a Jacobian by unfolding the residual matrix into a long vector and calculating a forward finite-difference.

33. The device of claim 32, wherein iteratively determining a vector of kinetic model parameter shifts that reduces a residual between a concentration profile computed from kinetic model parameters and a concentration profile obtained from the calibrated spectral data to provide revised kinetic model parameters includes using the Jacobian to generate updated kinetic model parameters for a subsequent iteration.

34. The device of claim 33, wherein iteratively determining a vector of kinetic model parameter shifts that reduces a residual between a concentration profile computed from kinetic model parameters and a concentration profile obtained from the calibrated spectral data to provide revised kinetic model parameters further includes calculating a revised residual matrix using updated kinetic model parameters and comparing to the previous residual matrix to determine whether to continue iterating or whether to set the updated kinetic model parameters as revised kinetic model parameters.

35. The device of claim 20, wherein transforming a concentration value obtained from second spectral data measured after a time interval using the revised kinetic model parameters to generate a calibrated concentration value of the analyte includes computing the dot product of a regression matrix calculated with revised kinetic model parameters and a concentration profile computed from second spectral data obtained after a time interval to generate a calibrated concentration value.

36. The device of claim 20, wherein the time interval is between 3 and 7 days.

37. The device of claim 20, wherein the time interval is between 1 and 3 days.

38. The device of claim 20, wherein the time interval is between 10 minutes and 24 hours.

39. A method of non-invasively measuring blood glucose concentration comprising:
obtaining a reference glucose concentration value from a blood sample of a patient;
obtaining first spectral data through a tissue layer of the patient;
calibrating the first spectral data using the reference glucose concentration value to generate calibrated spectral data;
iteratively determining a kinetic model parameter shift that reduces a residual between a glucose concentration profile computed from kinetic model parameters and a glucose concentration profile obtained from the calibrated spectral data to provide revised kinetic model parameters;
transforming a glucose concentration value obtained from second spectral data measured through the tissue layer of the patient after a time interval using the revised kinetic model parameters to generate a calibrated glucose concentration value.

40. The method of claim 39, wherein the kinetic model comprises a model of glucose transfer between blood and interstitial fluid.

41. The method of claim 39 wherein obtaining a reference glucose concentration value from a blood sample of a patient includes using a clinical glucose meter to analyze the blood sample.

42. The method of claim 39 wherein the first spectral data and second spectral data comprise one or more of Raman spectra, fluorescence spectra, or near infrared spectra.

43. The method of claim 39 further comprising:
transforming a glucose concentration value obtained from third spectral data measured through the tissue layer of the patient after a time interval using the revised kinetic model parameters to generate a calibrated glucose concentration value.

44. The method of claim 39, wherein the tissue layer is at least one of epidermis, cartilage, adipose, or nail.

45. A method of measuring an analyte, comprising:
measuring spectral data of an analyte in a biological sample;
using values for kinetic model parameters that represent analyte movement within the biological sample; and
transforming a concentration value obtained from the spectral data using the kinetic model parameters to generate a calibrated concentration value of the analyte.

46. The method of claim 45, further comprising selectively repeating a measurement of the analyte to determine an analyte concentration using the measured sample for at least 24 hours.

47. The method of claim 46, further comprising obtaining a second biological sample subsequent to the 24 hour period and measuring second spectral data of the second biological sample.

48. The method of claim 47, further comprising using the second spectral data to recalibrate subsequent transdermal measurements of analyte concentration.

49. The method of claim 45, wherein the analyte is glucose.

50. The method of claim 45 wherein Raman excitation light from a laser light source is coupled to a fiber optic probe, a distal end of the fiber probe, probe emitting the Raman excitation light is transmitted through the skin of the patient.

51. The method of claim 50 wherein the skin of the patient comprises an arm, a finger, and/or an earlobe of the patient.

52. The method of claim 45 further comprising processing data with a data processor.

53. The method of claim 45 further comprising withdrawing blood from a patient to provide the biological sample.

54. The method of claim 45 further comprising illuminating the sample with Raman excitation light, collecting from the sample with a fiber optic device, detecting the collected light to generate spectral data and processing the spectral data with a data processor.

55. The method of claim 54 further comprising collecting Raman light from tissue with a compound parabolic concentrator or a compound hyperbolic concentrator.

56. The method of claim 55 further comprising filtering the collected light.

57. The method of claim 45 wherein the measuring step comprises detecting Raman light with a photodetector.

58. The method of claim 57 further generating a plurality of Raman excitation wavelengths to illuminate tissue.

59. The method of claim 45 further comprising transmitting Raman excitation light through at tissue sample.

60. The method of claim 59 further comprising collecting light and a second side of the tissue sample with a collector coupled to a detector.
